(19) United States
(12) Patent Application Publication
(10) Pub. No.: US 2025/0352216 A1
(43) Pub. Date: Nov. 20, 2025

(54) POROUS MEMBRANES FOR VASCULAR OCCLUSION

(71) Applicant: VenaCore Inc., Wilmington, DE (US)

(72) Inventors: Michael Gabriel TAL, Tel Aviv (IL); Nir HOLTZMAN, Rishon Lezion (IL); Amit GREENER, Tel-Aviv (IL); Ronny WINSHTEIN, Ramat HaSharon (IL)

(21) Appl. No.: 19/194,642

(22) Filed: Apr. 30, 2025

Related U.S. Application Data
(63) Continuation of application No. PCT/US2023/078414, filed on Nov. 1, 2023.
(60) Provisional application No. 63/421,722, filed on Nov. 2, 2022.
(60) Provisional application No. 63/532,972, filed on Aug. 16, 2023.

(51) Int. Cl. A61B 17/12

(52) U.S. Cl. CPC A61B 17/12172; A61B 17/12145; A61B 17/1215; A61B 17/12177; A61B 2017/12054

(57) ABSTRACT

Vascular implants for vascular occlusion may define an enclosed or contained space. The vascular implant may comprise a porous membrane configured to adsorb and/or hold blood cells to contribute to blood coagulation and/or granulation in the porous membrane during a blood-filling time period.

### RELATED APPLICATIONS

This application is a continuation of International Application PCT/US2023/078414, filed Nov. 1, 2023, which application claims priority to U.S. Provisional Application 63/421,722 entitled Forming a Vascular Occluder in a Blood Vessel filed on Nov. 2, 2022 and U.S. Provisional Application 63/532,972 entitled Blood-Retaining Vascular Occluder filed on Aug. 16, 2023.

### FIELD

The present disclosure relates to vascular implants and methods for implanting vascular implants in a blood vessel, and more particularly, but not exclusively, to systems, devices and methods for forming a vascular occluder in blood vessels.

### BACKGROUND

Some medical procedures, such as embolization, involve occluding a blood vessel such as for reducing pressure on aneurysms, restricting a hemorrhage, or diminishing blood supply to tumors or growths in the body.

Vascular occlusion coils may be used for occluding voids in a patient vasculature using endovascular coiling and embolization techniques. Such coils have a minute spiral body usually made of soft metal and are sized and configured for delivery and implantation using a catheter. One or more coils are delivered in a single site, then manually curled and packed together at the target implantation site until forming a plug-like structure which serves to harvest coagulated blood adhering to its outer surface for gradually causing local occlusion and embolization.

Vascular plugs are a different type of mechanical embolization device commonly used for occluding a targeted portion of vein or artery with a relatively low-profile delivery and can be released in a controlled fashion. One type of vascular plug includes balloon-like expandable devices that aim to immediately block and seal the blood vessel lumen locally for preventing blood from flowing therethrough upon expansion. Another type of vascular plug includes expandable meshed (woven or braided, for example) devices that depend on natural blood coagulation, which can develop gradually over time on surfaces of foreign artifacts until potentially forming local embolization. While the first plug type is more prone to issues of unintentional implant migration and gradual physical and/or functional degradation, the second plug type requires substantial time until forming effective blocking and is associated with recanalization phenomenon by which openings are formed in the thrombus over time.

There is a need for improved vascular occlusion devices for achieving improved results such as in one or more of: reducing time from deploying to blocking; reducing likelihood of unintentional implant migration; preventing or postponing physical and/or functional degradation; and preventing or postponing recanalization.

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

The present disclosure relates to vascular implants and methods for implanting vascular implants in a blood vessel, and more particularly, but not exclusively, to systems, devices and methods for forming a vascular occluder in a blood vessel.

In certain embodiments, there is provided a vascular occluder, which can comprise: (a) a vascular implant configured to define an enclosed space configured to contain a volume of blood, the vascular implant is radially expandable for engaging an inner wall surface of a blood vessel; and (b) a porous membrane provided at least at the proximal end and/or the distal end of the container, configured to cover most or all luminal cross-sectional area of the blood vessel when the container engages the inner wall surface thereof.

In some embodiments, the porous membrane is configured to allow blood to flow therethrough into the container space during a predetermined blood-filling period after the container is radially expanded and engages the inner wall surface of the blood vessel, and then to prevent blood from flowing therethrough from the container space, thereby retaining the volume of blood in the container space when the volume of blood coagulates and/or granulates.

In some embodiments, the vascular occluder is configured to induce granulation tissue formation by the volume of blood retained in the container space.

In some embodiments, the vascular occluder is configured to induce blood coagulation in and/or on the porous membrane during the blood-filling period.

In some embodiments, the porous membrane is configured to adsorb and/or hold cells of blood passing therethrough associated with causing or contributing to blood coagulation and/or granulation, during the blood-filling period.

In some embodiments, the porous membrane is configured to adsorb blood-clotting proteins, such as fibrinogen and albumin.

In some embodiments, the porous membrane is configured to promote adhesion, activation, and aggregation of platelets.

In some embodiments, the porous membrane is configured as a thin fluid-permeable three-dimensional network structure.

In some embodiments, the porous membrane comprises a porous fluid-permeable random or aligned, three-dimensional network of polymeric microfibers and/or nanofibers.

In some embodiments, the three-dimensional network of polymeric microfibers and/or nanofibers is configured with average fiber diameter smaller than about 5 micrometer, optionally smaller than about 2 micrometer, and/or average pore size smaller than about 50 micrometer and/or within a range of about 1 micrometer to about 50 micrometer.

In some embodiments, the porous membrane is formed by way of electrospinning.

In some embodiments, the porous membrane is provided both at the proximal end and at the distal end of the container.

In some embodiments, most or all surface of the container is connected to, coated over, or impregnated with the porous membrane.

In some embodiments, the vascular occluder further comprising a container expanding and/or anchoring element configured to expand the container to over the local inner diameter of the blood vessel and/or to maintain the container radially pressed against the inner wall surface of the blood vessel for anchoring the container thereto.

In some embodiments, the container expanding and/or anchoring element is configured for selective filling of the container space for affecting radial expansion and/or anchoring of the container.

In some embodiments, the container expanding and/or anchoring element comprises a flexible member having an elastically stretchable three-dimensional frame structure.

In some embodiments, the container includes a flexible tubular wall that is optionally meshed, woven, braided or perforated.

In some embodiments, the flexible tubular wall is formed of metallic material such as Ni—Ti alloy, optionally in a form of braided wire.

In some embodiments, the predetermined blood-filling period is smaller than about 90 seconds, optionally particularly smaller than about 60 seconds, optionally particularly smaller than about 30 seconds, optionally particularly smaller about 10 seconds, or optionally particularly smaller about 5 second.

In some embodiments, the predetermined blood-filling period is smaller than a minimally achievable result in a local activated clotting time (ACT) type test of coagulation.

In some embodiments, the container space is undivided so as to promote formation of a single coagulated or granulated mass sized to fill most or all of the container space for occluding the blood vessel.

In certain embodiments, there is provided a method for occluding a blood vessel. The method may comprise: positioning the vascular occluder in a blood vessel; expanding the device such that the device engages a wall of the blood vessel; allowing blood to flow through the porous membrane to fill the container space until the porous membrane is at least 75% clogged with blood cells and/or coagulated blood preventing blood from flowing therethrough, so as to retain a volume of blood in the container space; and retaining the volume of blood in the container space during coagulation and/or granulation thereof into a single coagulated or granulated mass sized to fill most or all of the container space for occluding the blood vessel.

In some embodiments, the porous membrane is configured to completely clog within the predetermined blood-filling period less than about 90 seconds.

In some embodiments, the expanding is configured in size and/or magnitude sufficient for causing local inflammation such as by way of stretching the blood vessel wall and/or preventing oxygenation thereof.

In some embodiments, the expanding includes filling the container space with a three-dimensional frame structure.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIGS. 1A-1D schematically illustrate exemplary scenarios representing steps in a method for forming a vascular occluder in a blood vessel using a vascular occlusion coil, according to some embodiments;

FIGS. 2A-2B schematically illustrate a cross-sectional view of an exemplary vascular occluder before and after activation, according to some embodiments;

FIGS. 3A-3F schematically illustrate exemplary scenarios representing steps in a method for implanting the second exemplary vascular occluder of FIGS. 2A-2B in a blood vessel, according to some embodiments;

FIGS. 4A-4B schematically illustrate a cross-sectional view of a third exemplary vascular occluder before and after activation, according to some embodiments; and

FIGS. 5A-5D schematically illustrate exemplary scenarios representing steps in a method for extracting the third exemplary vascular occluder of FIGS. 4A-4B from a blood vessel, according to some embodiments.

### DETAILED DESCRIPTION

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

Certain embodiments relate to vascular implants and methods for implanting vascular implants in a blood vessel, and more particularly, but not exclusively, to systems, devices and methods for forming a vascular occluder in blood vessels. In some embodiments, there is provided a system for forming a vascular occluder that includes a vascular occlusion coil and means for deploying and shaping the coil in a tubular lumen of a target blood vessel. In some embodiments, such means may include a core member configured to shape (e.g., impose a shape of) the vascular occlusion coil from an inner passage or space enclosed by the spiral body of the vascular occlusion coil, and/or a shaped or shapeable container configured to shape (e.g., impose a shape of) the vascular occlusion coil from its surrounding space formed by the container, to which the coil is inserted.

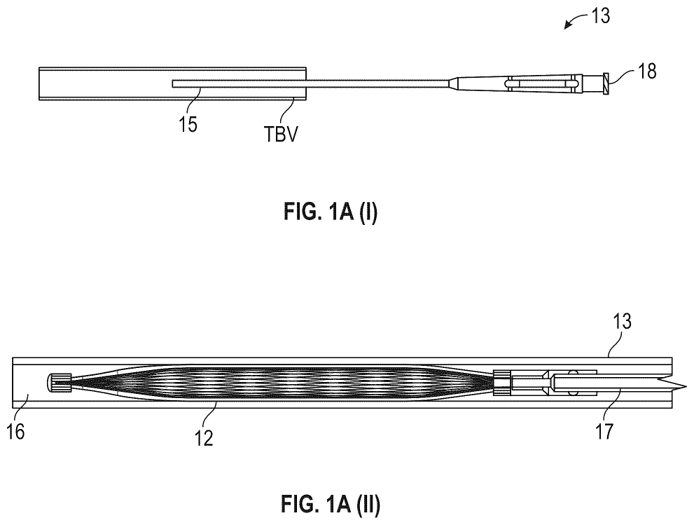

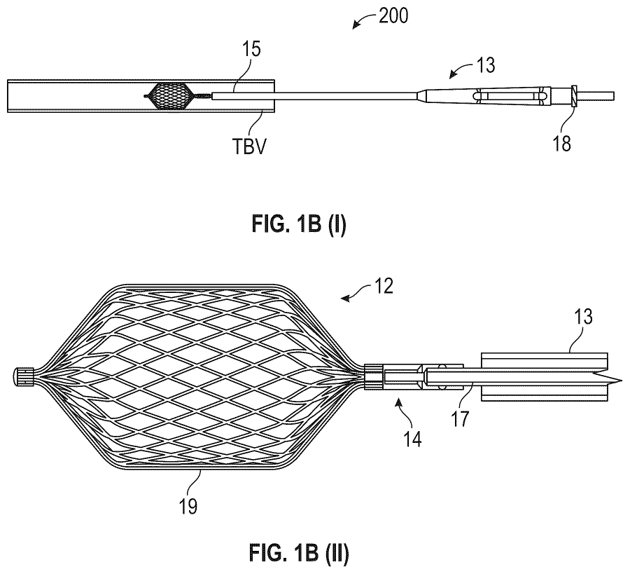

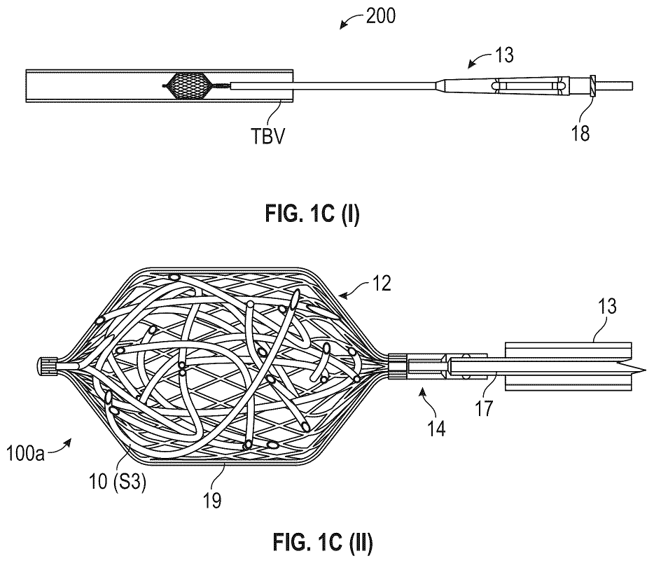

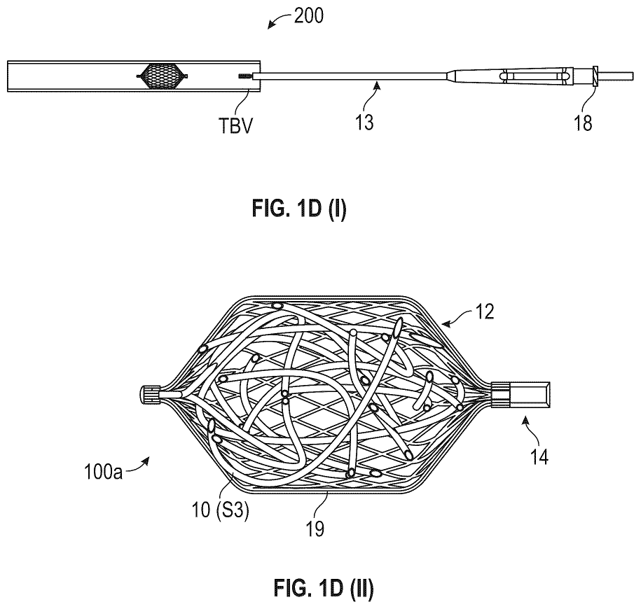

FIGS. 1A-1D schematically illustrate exemplary scenarios representing steps in a method for forming a vascular occluder 100a in a target blood vessel TBV using a vascular occlusion coil 10. A system 200 for forming vascular occluder 100a includes coil 10, a container 12, a catheter 13 (e.g., a single-lumen catheter, optionally a microcatheter), and a coil dispenser 14.

FIG. 1A(I) shows a first scenario wherein catheter 13 with a distal portion 15 thereof is provided in a lumen of target blood vessel (TBV) (catheter 13 is shown in a side view, and blood vessel TBV is shown in a side cross-sectional view for ease of description). FIG. 1A(II) illustrates catheter distal portion 15 in an enlarged side cross-sectional view. Container 12 is provided and/or deliverable in a crimped or radially compacted configuration in a lumen 16 of catheter 13 (optionally particularly in distal portion 15). Container 12 is pushable and optionally releasably connected to an elongated pusher 17 (such as by way of threading or snap-locking) which optionally extends along lumen 16 such that a proximal end thereof is manipulatable by a user via a proximal portion 18 of catheter 13.

FIG. 1B(I) shows a second scenario wherein container 12 is pushed via distal portion 15 into the lumen of target blood vessel TBV (container 12 and catheter 13 are shown in side view, and blood vessel TBV is shown in a side cross-sectional view for ease of description). FIG. 1B(II) illustrates container 12 connected to pusher 17 emerging via distal portion 15 in an enlarged side cross-sectional view. Container 12 is allowed to elastically expand creating a contained space, optionally until engaging a side wall 19 thereof with inner wall surface of target blood vessel TBV. In some embodiments, the user can choose container 12 of a specific size and/or shape in accordance with some fitting consideration thereof in target blood vessel TBV. Container 12 may be formed as a tubular or other shaped structure by way of one or more wires which may be braided or otherwise arranged and coupled as is known in the art, optionally metal wires (e.g., Ni—Ti or Co—Cr alloy wires), although it can be made by other materials, optionally non-stretching wires, such as nylon, polyester, cotton, polypropylene or aramid. As will be described further below, the container 12 is advantageously fully or partially covered or impregnated with another material, optionally in a form of coating layers or a membrane, however, side wall 19 is configured permeable to liquids for allowing or facilitating blood from target blood vessel TBV to flow therethrough into container 12. Optionally, additionally or alternatively, one or more openings on or next to side wall 19 are configured to allow inflow of blood into container and/or outflow of blood from container 12. In some embodiments, container 12 is configured such that it cannot be effectively inflated by gas and/or liquid, and/or that it is configured to self-expand while allowing fluid flow thereinto.

FIG. 1C(I) shows a third scenario wherein vascular occlusion coil 10 is completely dispensed within container 12 (container 12 and catheter 13 are shown in side view, and blood vessel TBV is shown in a side cross-sectional view for ease of description). FIG. 1C(II) illustrates container 12 connected to pusher 17 emerging via distal portion 15 in an enlarged side cross-sectional view. Vascular occlusion coil 10 is arranged into a three-dimensional tertiary structure (which may be cocoon-like) denoted S3 in FIG. 1C(II) by being pushed gradually through coil dispenser 14 with arced segments thereof engaging the shaped covering of container 12 forcing it to deform as such. Coil 10 may be advanced using pusher 17 or through pusher 17, optionally using other means. In some embodiments, once fully deployed in container 12, vascular occlusion coil 10 forces container 12 to expand laterally and/or compress axially, thereby increasing anchoring force or pressure against walls of target blood vessel TBV. FIGS. 1D(I) and (II) similarly illustrate system 200 after vascular occluder 100a, formed by container 12 filled with coil 10 in the tertiary structure, is disconnected from pusher 17 and after catheter 13 is removed from blood vessel TBV, leaving vascular occluder in place.

FIGS. 2A-2B schematically illustrate a cross-sectional view of another exemplary vascular occluder 100b before and after activation. Vascular occluder 100b includes a container 31. The container may be substantially as illustrated above with reference to FIGS. 1A-1D, and may be implanted by the same or similar methods as described above. and a porous (temporarily porous in some embodiments as set forth in detail below) membrane 32 provided over some or all of the outer surface of the container 31. In some embodiments, membrane 32 may be provided only over the areas of the proximal end 33 and the distal end 34 of container 31. Container 31 encloses a container space 25 configured to contain a volume of blood. Container 31 is radially expandable for engaging an inner wall surface of a blood vessel. Similar to the embodiments described above, container 31 includes a side wall that is optionally meshed, woven, braided, or perforated, and is optionally formed of metallic material such as Ni—Ti alloy.

Porous membrane 32 is configured to cover most or all lumen cross-sectional area of the blood vessel when container 31 engages inner surface of the blood vessel wall. Porous membrane 32 is configured to allow blood to flow therethrough into container space 25 during a predetermined blood-filling period, after the container is radially expanded, and then to prevent blood from flowing therethrough from container space 25, thereby retaining the volume of blood in container space 25 when the volume of blood coagulates and/or granulates. As shown, container space 25 may be undivided so as to promote formation of a single coagulated or granulated mass sized to fill most or all its volume. The predetermined blood-filling period is optionally smaller than about 90 seconds, optionally particularly smaller than about 60 seconds, optionally particularly smaller than about 30 seconds, optionally particularly smaller than about 10 seconds, or optionally particularly smaller about 5 seconds. Additionally or alternatively, the predetermined blood-filling period may be smaller than a minimally achievable result in a local activated clotting time (ACT) type test of coagulation, depending on patient-specific reaction to use of anticoagulants.

In some embodiments, vascular occluder 100b is configured to induce blood coagulation in and/or on porous membrane 32 during the blood-filling period, and/or to induce granulation tissue formation by the volume of blood retained in container space 25 after the blood-filling period. In some embodiments, porous membrane 32 is configured to adsorb and/or hold cells of blood passing therethrough associated with causing or contributing to blood coagulation and/or granulation, during the blood-filling period, including but not limited to blood-clotting proteins, such as fibrinogen and albumin. Porous membrane 32 is optionally configured to promote adhesion, activation, and aggregation of platelets.

In some embodiments, porous membrane 32 is configured as a thin fluid-permeable three-dimensional network structure, optionally comprising a porous fluid-permeable random or aligned, three-dimensional network of polymeric microfibers and/or nanofibers. The three-dimensional network of polymeric microfibers and/or nanofibers is configured with average fiber diameter smaller than about 5 micrometer, optionally smaller than about 2 micrometer, and/or average pore size smaller than about 50 micrometer and/or within a range of about 1 micrometer to about 50 micrometer. In some embodiments, porous membrane 32 is formed by electrospinning ultrafine fibers over a revolving mandrel covered with container 31, such that most or all surface of container 31 is connected to, coated over, or impregnated with porous membrane 32.

In some embodiments, porous membrane 32 is configured as a porous electrospun microfiber and/or nanofiber membrane, comprising of a three-dimensional network of polymeric microfibers and/or nanofibers made in electrospinning. In some such embodiments, porous membrane 32 has a porous and fluid-permeable sponge-like structure that is characterized by its high porosity, large specific surface area, small pore size, good channel connectivity, and ease of functional modification. In some embodiments, porous membrane is configured as a 3D electrospun fibrous sponge constructed by a 3D reconstruction of electrospun fiber membranes. In some such embodiments, fibrous membrane 32 is made via electrospinning, homogenization, shaping and thermal crosslinking, and exhibits high porosity, water absorption and compression resilience.

Container 31 may be configured to self-expand to a certain state when under relatively small resistance, such as within a lumen of a blood vessel however without significant strength to locally expand or stretch radially the blood vessel wall. Vascular occluder 100b may further include a container expanding and/or anchoring element 36 configured to expand container 31 to over the local inner diameter of a host blood vessel and/or to maintain container radially pressed against the inner wall surface of the blood vessel for anchoring container 31 thereto. Container expanding and/or anchoring element 36 may include a flexible member having an elastically stretchable a three-dimensional frame structure configured for selective filling of container space 25 for affecting radial expansion and/or anchoring of container 31. In some embodiments, the container expanding and/or anchoring element 36 may comprise an occlusion coil in accordance with the various occlusion coil embodiments described herein.

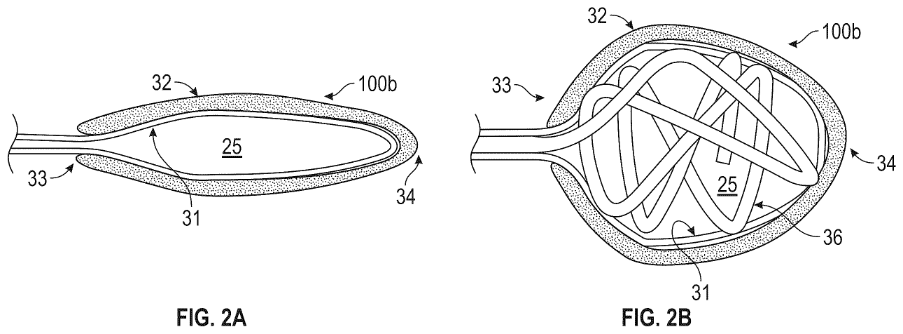

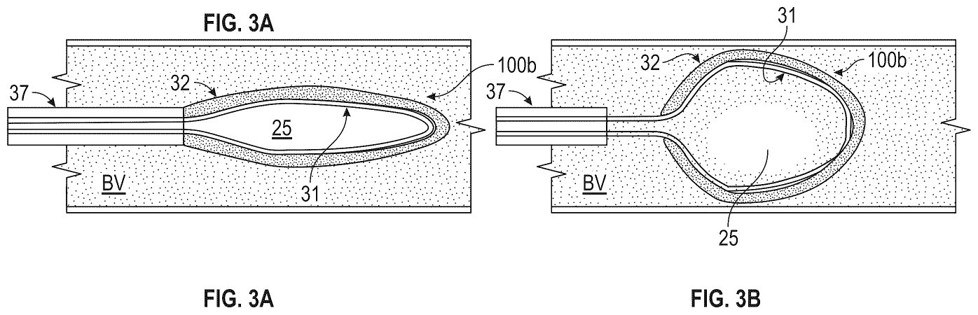

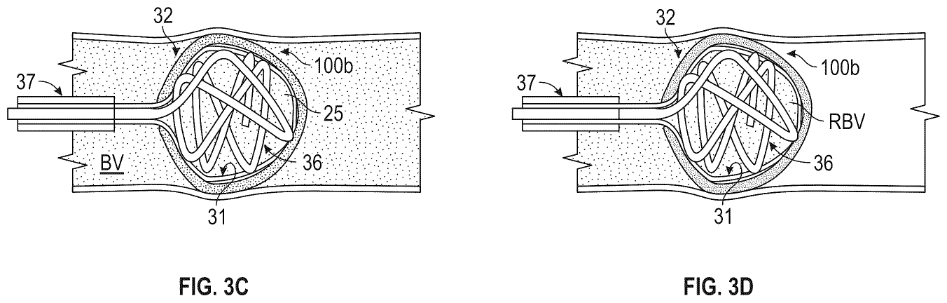

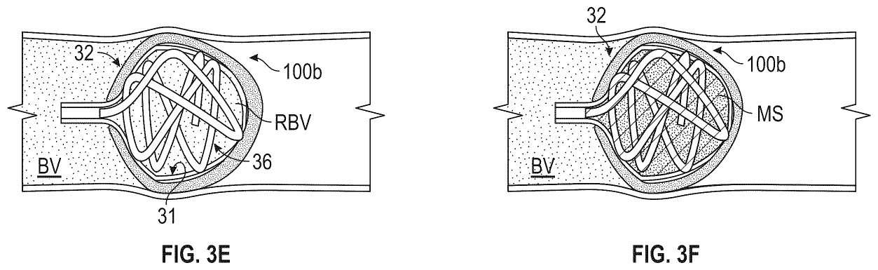

FIGS. 3A-3F schematically illustrate exemplary scenarios representing steps in a method for implanting vascular occluder 100b of FIGS. 2A and 2B in blood vessel BV. A distal end of a delivery device 37 is first introduced into a chosen location within blood vessel BV, then vascular occluder 100b can be extracted out of delivery device 37 such as by holding (e.g., with a pusher) vascular occluder 100b in place while pulling delivery device 37 proximally until uncovering vascular occluder 100b in blood vessel BV. Further positioning can be performed as needed as long as delivery device 37 is connected to vascular occluder 100b. FIG. 3A shows vascular occluder 100c partially extracted from distal end of delivery device 37. At this stage, blood may begin to penetrate through porous membrane 32 into container space 25 however optionally in insignificant volume. FIG. 3B shows vascular occluder 100b fully uncovered and extracted from delivery device 37 such that it can self-expand up to a certain (not maximal) degree within lumen of blood vessel BV. At this stage more blood is accumulated in container space 25 and can flow through porous membrane 32 into and from container space 25.

As shown in FIG. 3C, container expanding and/or anchoring element 36 is then introduced into container space 25 via delivery device 37 and through a dedicated opening in porous membrane 32. Element 36 is delivered in an elastically stretched form and is then allowed to elastically regain a more volume expanded three-dimensional form in container space 25, however it is still restricted from forming its maximally expanded unstressed three-dimensional frame structure. Therefore, element 36 applies a continuous radial or volumetric pressure on container 31 thereby forcing it to further expand radially together with porous membrane 32 against the blood vessel wall. In some embodiments, element 36 is configured to apply sufficient pressure through container 31 onto blood vessel wall such that the latter expands laterally, and in some particular embodiments this causes sufficient stretching of blood vessel wall and/or diminishes oxygenation thereof so as to cause or induce local inflammation. In some embodiments, this additional expansion under stress of container 31 increases pressure difference which causes suction of blood into container space 25 via porous membrane 32 until substantially or completely filling container space 25 with blood.

FIG. 3D illustrates the situation of the deployed vascular occluder 100b in blood vessel BV after the blood-filling period, optionally within a few seconds to about 90 seconds. As shown, after the blood-filling period, fibrous membrane 32 can be substantially or completely clogged, such that blood is prevented from flowing therethrough into and out of container space 25. This results in blocking flow of blood in blood vessel BV and retaining a certain volume of blood RBV in container space 25 which is subjected to natural coagulation and/or granulation process. As shown in FIG. 3E, delivery device 37 can be disconnected from vascular occluder 100b and removed from blood vessel BV, and this can be executed before or after fibrous membrane 32 is substantially or completely clogged.

FIG. 3F shows vascular occluder 100b after several days, weeks, or months following implantation thereof in blood vessel BV, after natural coagulation and/or granulation of retained blood volume RBV. In some embodiments, vascular occluder 100b is configured to allow, cause or promote natural formation of a single formed mass MS of granulation tissue and/or thrombosis filling most or all container space 25 occluding blood vessel BV. In some embodiments, causing inflammation in blood vessel BV, such as by way of stretching the blood vessel wall and/or preventing oxygenation thereof with element 26, causes, induces, or promotes particularly the formation of granulation tissue within entire volume of container space 25.

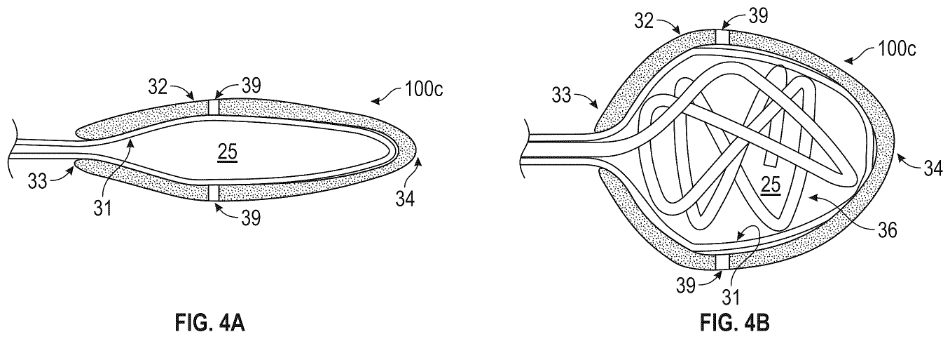

FIGS. 4A-4B schematically illustrate a cross-sectional view of a third exemplary vascular occluder 100c before and after activation. Vascular occluder 100c may be similar or identical in most or all structural and/or functional embodiments to vascular occluder 100b other than that it also includes one or more blood extraction openings 39 in porous membrane 32. Similar to vascular occluder 100b, vascular occluder 100c includes container 31, porous membrane 32, proximal end 33, distal end 34, container space 25, and container expanding and/or anchoring element 36, and is optionally releasably connectable to delivery device 37. Blood extraction openings 39 are configured to allow ejection of blood from container space 25 to the host blood vessel when container 31 is pressurized such as by extraction thereof by external force. This embodiment can be found advantageous for example when, after initial deployment and substantial clogging of porous membrane 22, a user wishes to remove or reposition vascular occluder 100c. In some embodiments, blood extraction openings 39 are provided at lateral portions configured for engaging blood vessel wall when container 31 is expanded using element 36, such that openings 39 are covered by the blood vessel wall when container 31 is pressed against it. In some embodiments, blood extraction openings 39 are configured as through holes, cuts, septum valves or pressure relief valves. In some embodiments, blood extraction openings 39 are about 1 mm or less, optionally 0.5 mm or less in diameter.

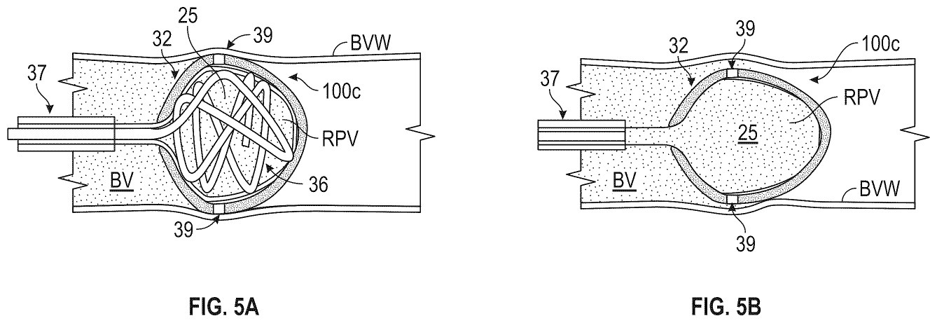

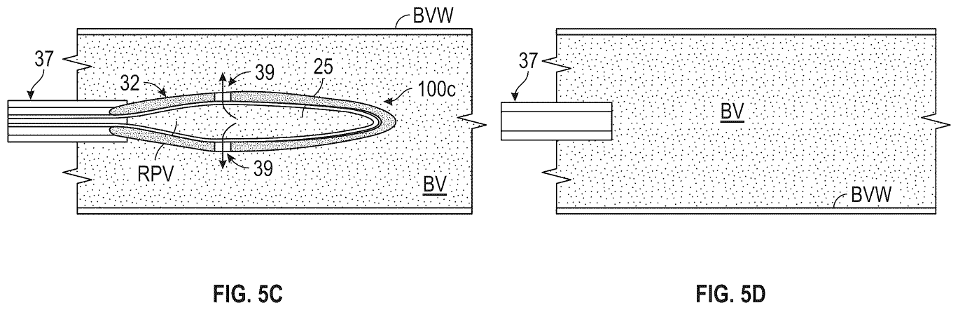

FIGS. 5A-5D schematically illustrate exemplary scenarios representing steps in a method for extracting vascular occluder 100c from blood vessel BV. FIG. 5A represents a possible exemplary scenario in which vascular occluder 100d is already deployed in blood vessel BV, whereas container space 25 is substantially or completely filled with retained blood volume RBV, and fibrous membrane 32 is substantially or fully clogged (e.g., over than about 75% clogged, optionally over 90% clogged) with coagulated blood (e.g., covering it and/or built up across its thickness such as including within its pores) so as to retain the already present volume of blood RBV in container space 25. Since the container 31 presses against wall BVW of blood vessel BV and blood extraction openings 39 are clogged or blocked by the blood vessel wall BVW, the retained blood volume RBV cannot escape container space 25.

FIG. 5B represents a possible scenario following a decision to remove or reposition vascular occluder 100c after the earlier scenario shown in FIG. 5A, in which a first step would be to remove element 36 from occupying container space 25 that functions to expand container 31 to press against blood vessel wall BVW. This may cause immediate initial contraction of container 31 and/or uncovering of blood extraction openings 39. Depending on one or more variables, such as the effective opened area of blood extraction openings 39, the internal pressure within container space 25, and/or the viscosity of the retained volume of blood RBV within container space 25, blood may already begin to infiltrate from container space 25 via openings 39 at this stage.

An ejection of blood from container space 25 via openings 39 in substantial magnitude can be accomplished when vascular occluder 100c is pulled back into lumen of delivery device 37 (or another device or instrument), as shown in FIG. 5C. This process causes an extraction of vascular occluder 100c and forces retained blood volume RBV to eject from container space 25 into blood vessel BV through the blood extraction openings 39, due to the increased internal pressure in container space 25 as derived from the pulling force applied to container 31. Substantial or complete emptying of the retained blood RBV from container space 25 can be accomplished once vascular occluder is fully withdrawn into delivery device 37, as suggested in FIG. 5D.

### General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C.’.

When applied to a numerical value, the term ‘about’, as used herein, refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment, may also be illustratively described and presented in the context or format of a plurality of separate embodiments.

Although the invention has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

1. A vascular occluder, comprising:
a vascular implant configured to create an enclosed space when engaged with an inner wall surface of a blood vessel, wherein the enclosed space is configured to contain a volume of blood, wherein the vascular implant is radially expandable for engaging the inner wall surface of the blood vessel; and
a porous membrane provided on some or all of the vascular implant and configured to cover most or all luminal cross-sectional area of the blood vessel when the vascular implant is engaged with the inner wall surface thereof.

2. The vascular occluder of claim 1, wherein the porous membrane is configured to allow blood to flow therethrough into the enclosed space during a predetermined blood-filling period after the vascular implant is radially expanded and engages the inner wall surface of the blood vessel, and then to prevent blood from flowing therethrough from the enclosed space, thereby retaining the volume of blood in the enclosed space as the volume of blood coagulates and/or granulates.

3. The vascular occluder of claim 2, configured to induce granulation tissue formation by the volume of blood retained in the enclosed space.

4. The vascular occluder of claim 2, configured to induce blood coagulation in and/or on the porous membrane during the blood-filling period.

5. The vascular occluder of claim 2, wherein the porous membrane is configured to adsorb and/or hold cells of blood passing therethrough associated with causing or contributing to blood coagulation and/or granulation, during the blood-filling period.

6. The vascular occluder of claim 2, wherein the porous membrane is configured to adsorb blood-clotting proteins, such as fibrinogen and albumin.

7. The vascular occluder of claim 2, wherein the porous membrane is configured to promote adhesion, activation, and aggregation of platelets.

8. The vascular occluder of claim 2, wherein the porous membrane is configured as a thin fluid-permeable three-dimensional network structure.

9. The vascular occluder of claim 2, wherein the porous membrane comprises a porous fluid-permeable random or aligned, three-dimensional network of polymeric microfibers and/or nanofibers.

10. The vascular occluder of claim 9, wherein the three-dimensional network of polymeric microfibers and/or nanofibers is configured with average fiber diameter smaller than about 5 micrometer, optionally smaller than about 2 micrometer, and/or average pore size smaller than about 50 micrometer and/or within a range of about 1 micrometer to about 50 micrometer.

11. The vascular occluder of claim 10, wherein the porous membrane is formed by way of electrospinning.

12. The vascular occluder of claim 2, wherein the porous membrane is provided at least at both at the proximal end and at the distal end of the vascular implant.

13. (canceled)

14. The vascular occluder of claim 2, wherein the predetermined blood-filling period is smaller than about 90 seconds, optionally particularly smaller than about 60 seconds, optionally particularly smaller than about 30 seconds, optionally particularly smaller about 10 seconds, or optionally particularly smaller about 5 second.

15. The vascular occluder of claim 2, wherein the predetermined blood-filling period is smaller than a minimally achievable result in a local activated clotting time (ACT) type test of coagulation.

16. The vascular occluder of claim 2, wherein the vascular occluder further comprises a container with an expanding and/or anchoring element configured to expand the container to over cover the local inner diameter of the blood vessel and/or to maintain the container radially pressed against the inner wall surface of the blood vessel for anchoring the container thereto.

17. (canceled)

18. The vascular occluder of claim 16, wherein the container expanding and/or anchoring element comprises a flexible member having an elastically stretchable three-dimensional frame structure.

19. (canceled)

20. The vascular occluder of claim 16, wherein the container expanding and/or anchoring element comprises a vascular occlusion coil, and wherein arced segments of the vascular occlusion coil engage a side wall of the container.

21. (canceled)

22. (canceled)

23. A method for occluding a blood vessel, the method comprising:
positioning a vascular occluder according to claim 2 in a blood vessel;
radially expanding the vascular occluder such that the vascular occluder engages a wall of the blood vessel;
allowing blood to flow through the porous membrane to fill the enclosed space until the porous membrane is at least 75% clogged with blood cells and/or coagulated blood preventing blood from flowing therethrough, so as to retain a volume of blood in the enclosed space; and
retaining the volume of blood in the enclosed space during coagulation and/or granulation thereof into a single coagulated or granulated mass sized to fill most or all of the enclosed space for occluding the blood vessel.

24. The method according to claim 23, wherein the porous membrane is configured to completely clog within the predetermined blood-filling period less than about 90 seconds.

25. The method according to claim 23, wherein the expanding is configured in size and/or magnitude sufficient for causing local inflammation such as by way of stretching the blood vessel wall and/or preventing oxygenation thereof.

26. (canceled)
